(19) United States
(12) Patent Application Publication
(10) Pub. No.: US 2006/0030852 A1
(43) Pub. Date: Feb. 9, 2006

(54) Anchoring system for fixing objects to bones

(76) Inventor: Lionel C Sevrain, West Palm Beach, FL (US)

Correspondence Address: BURNS & LEVINSON LLP, 1030 15TH STREET NW, SUITE 300, WASHINGTON, DC, 20005-1501, US

(21) Appl. No.: 10/535,054

(22) PCT Filed: Nov. 13, 2003
(86) PCT No.: PCT/CA03/01722

§ 371 Date: May 13, 2005

(30) Foreign Application Priority Data
Nov. 13, 2002 (CH) 1902/02

(51) Int. Cl. A61B 17/86; A61B 17/80; A61B 17/70

(52) U.S. Cl. 606/73; 606/61; 623/23.44; 606/69

(57) ABSTRACT

An anchoring system for mounting an object (O) to a bone (B), comprising first (M) and second (F) anchoring members each having proximal and distal ends. The proximal ends are adapted to hold the object (O) to the bone (B) and are spaced from each other with the first and second anchoring members (M,F) converging from the proximal ends towards the distal ends. The anchoring members (M, F) are adapted to be connected to each other in the bone and distally of the proximal ends. There are provided at least two first anchoring members (M) each adapted to be connected to the second anchoring member (F). The second anchoring member (F) and each of the first anchoring members (M) define co-operating engagement means for connecting each first anchoring member (M) to the second anchoring member (F). Typically, the second anchoring member (F) defines a number of openings (A) each adapted to receive a first anchoring member (M). The first anchoring members (M) can extend coplanarly when connected to the second anchoring member (F), or can extend in at least two different planes.

### BACKGROUND OF THE INVENTION

(a) Field of the Invention

The present invention relates to devices for attaching various objects, such as prostheses or implants, to bones, including for anchoring spinal instrumentations to vertebrae of the human rachis and for fixing broken bones.

(b) Description of Prior Art

U.S. Pat. No. 5,366,455 issued to Dove et al. on Nov. 22, 1994, U.S. Pat. No. 5,672,175 issued to Martin on Sep. 30, 1997, U.S. Pat. No. 5,733,284 issued to Martin on Mar. 31, 1998 and U.S. Pat. No. 5,437,672 issued to Alleyne on Aug. 1, 1995 disclose devices for anchoring various supports, e.g. spinal orthoses, to the rachis, these devices being adapted to obviously extend outwardly of the spinous process or canal and thus of the spinal cord.

U.S. Pat. No. 5,800,433 issued to Benzel et al. on Sep. 1, 1998 and U.S. Pat. No. 5,954,722 issued to Bono on Sep. 21, 1999 teach anchoring systems having screws which are angled such as to converge towards each other.

U.S. Pat. No. 5,904,683 issued to Pohndorf et al. on May 18, 1999 and U.S. Pat. No. 5,980,523 issued to Jackson on Nov. 9, 1999 disclose anterior cervical vertebral stabilising devices held in place by various types of screws.

To try preventing the screws from loosening, various systems have been used, such as directing the screws along different orientations (e.g. diverging or converging); providing a locking mechanism on the screw (e.g. counter-nut); modifying the screw's thread (height and depth); engaging each screw to two tissues having different densities; etc.

### SUMMARY OF THE INVENTION

It is therefore an aim of the present invention to provide a novel anchoring system for securing various objects to bones, such as spinal devices or instrumentations to the rachis and plates or other to broken bones.

It is also an aim of the present invention to provide an anchoring system well adapted to prevent a loosening thereof over time.

Therefore, in accordance with the present invention, there is provided an anchoring system for a bone, comprising first and second anchoring members each having proximal and distal ends, said proximal ends being spaced from each other with said first and second anchoring members converging from said proximal ends towards said distal ends, said anchoring members being adapted to be connected to each other in the bone and distally of said proximal ends, wherein there are provided at least two said first anchoring members each adapted to be connected to said second anchoring member.

Also in accordance with the present invention, there is provided an anchoring system for mounting an object to a bone, comprising first and second anchoring members each having proximal and distal ends, said proximal ends being adapted to hold the object to the bone, said proximal ends being spaced from each other with said first and second anchoring members converging from said proximal ends towards said distal ends, said anchoring members being adapted to be connected to each other in the bone and distally of said proximal ends, wherein there are provided at least two said first anchoring members each adapted to be connected to said second anchoring member.

### BRIEF DESCRIPTION OF THE DRAWINGS

Having thus generally described the nature of the invention, reference will now be made to the accompanying drawings, showing by way of illustration a preferred embodiment thereof, and in which:

FIG. 1 is a schematic cross-sectional plan view of a bridging plate mounted to a lumbar vertebra using an anchoring system in accordance with the present invention;

FIG. 2 is a schematic anterior perspective view of a bridging plate mounted to a pair of cervical vertebra using the anchoring system in accordance with the present invention;

FIG. 3 is a schematic view of an anchoring system in accordance with the present invention used for instance to hold an object, such as bridging plate, to a bone in a configuration similar to that of FIGS. 1 and 2;

FIG. 4 is a schematic view similar to FIG. 3 but wherein the male anchor engages the female anchor at a different location than in FIG. 3;

FIG. 5 is a schematic view of an anchoring system also in accordance with the present invention and similar to that of FIGS. 1 to 4, but herein two or more male anchors, extending in a same plane, engage a same female anchor;

FIG. 6 is a schematic view of an anchoring system in accordance with the present invention similar to that of FIG. 5 that is used to stabilize a broken bone, e.g. femur;

FIG. 6a is an enlarged view of part of FIG. 6 delimited by broken lines;

FIG. 7 is a schematic view of an anchoring system in accordance with the present invention similar to that of FIG. 5, but wherein the male anchors extend in more than one plane;

FIG. 8 is a schematic view of an anchoring system in accordance with the present invention similar to that of FIG. 7 that is used to stabilize a broken bone, e.g. femur;

FIG. 9 is a schematic view of the anchoring system of FIG. 5 but used to stabilize a broken bone and in a different manner than shown in FIGS. 6 and 8;

FIG. 10 is a schematic view of a further anchoring system in accordance with the present invention wherein one anchor is of a hybrid configuration in being able to act both as a female and a male anchor;

FIG. 11 is a schematic view of a first arrangement for permitting the male and female anchors of the anchoring system to be connected together;

FIGS. 12a and 12b are schematic views that show respectively installation and engagement positions of a second arrangement for permitting the male and female anchors of the anchoring system to be connected together; and

FIGS. 13a, 13b and 13c are schematic views that show two successive installation positions and one engagement position of a third arrangement for permitting the male and female anchors of the anchoring system to be connected together.

### DESCRIPTION OF THE PREFERRED EMBODIMENTS

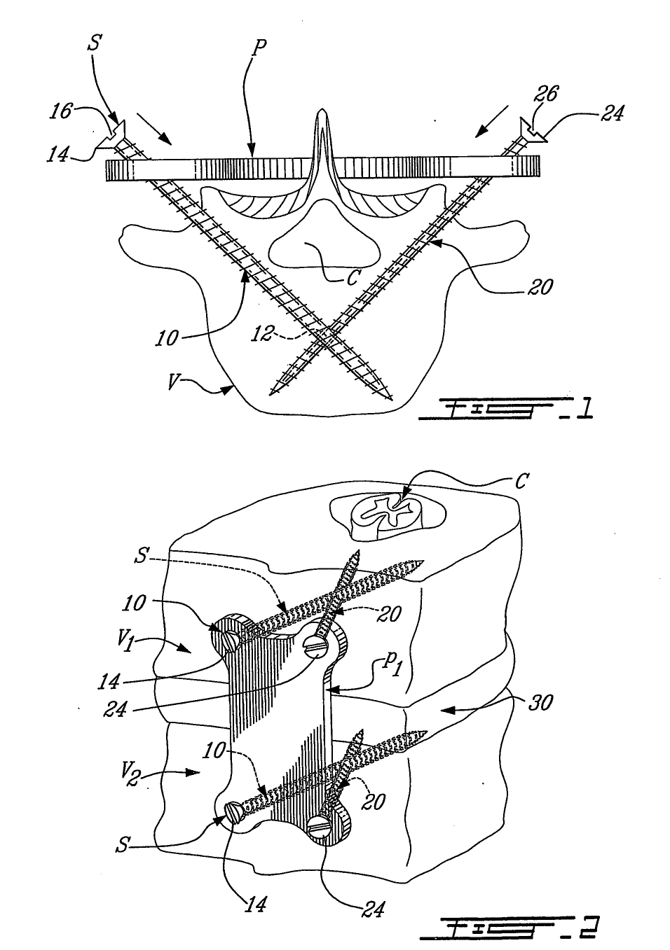

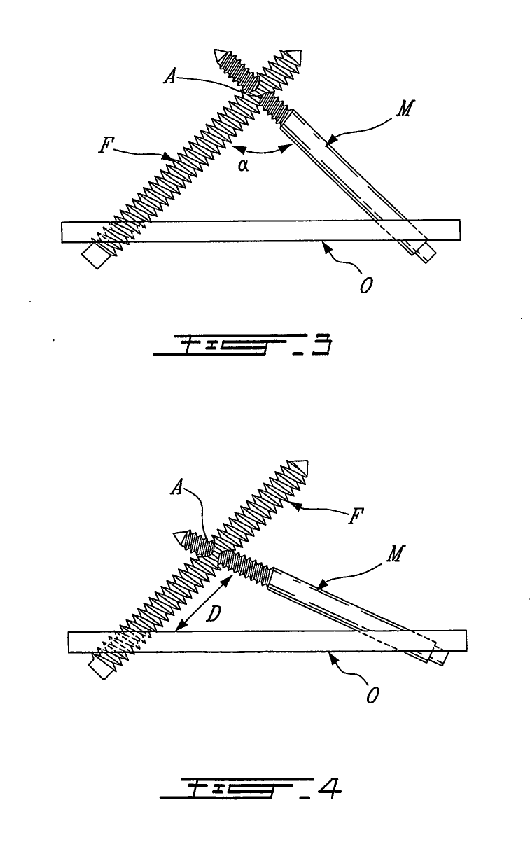

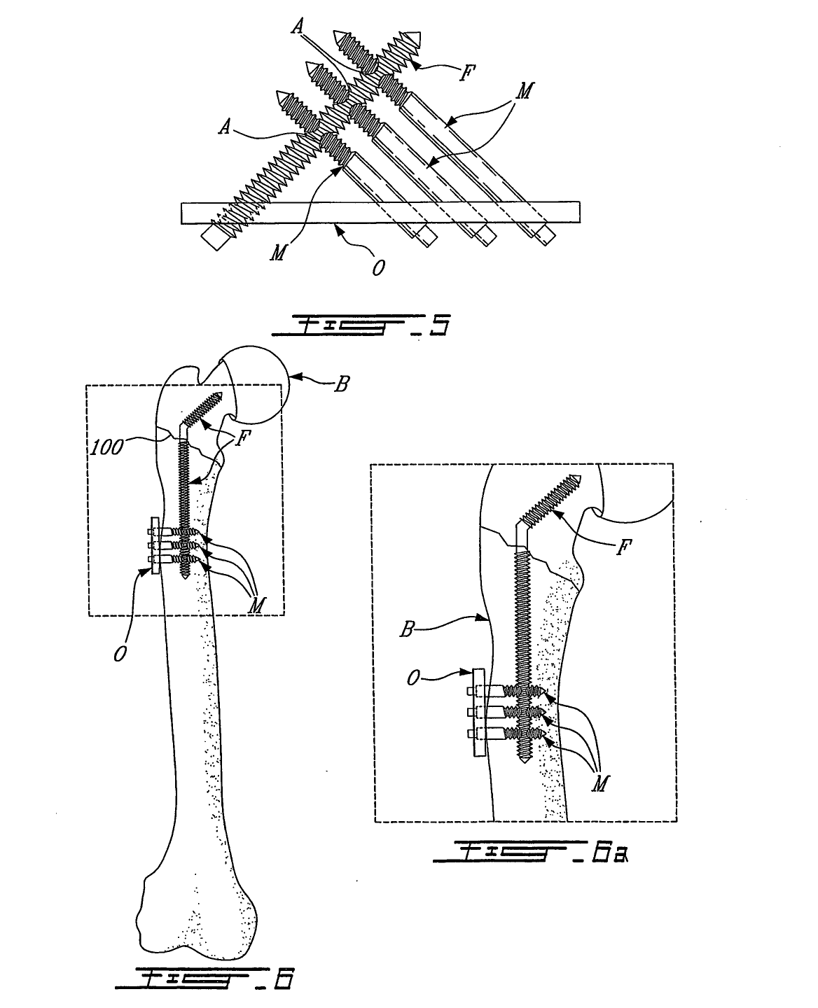

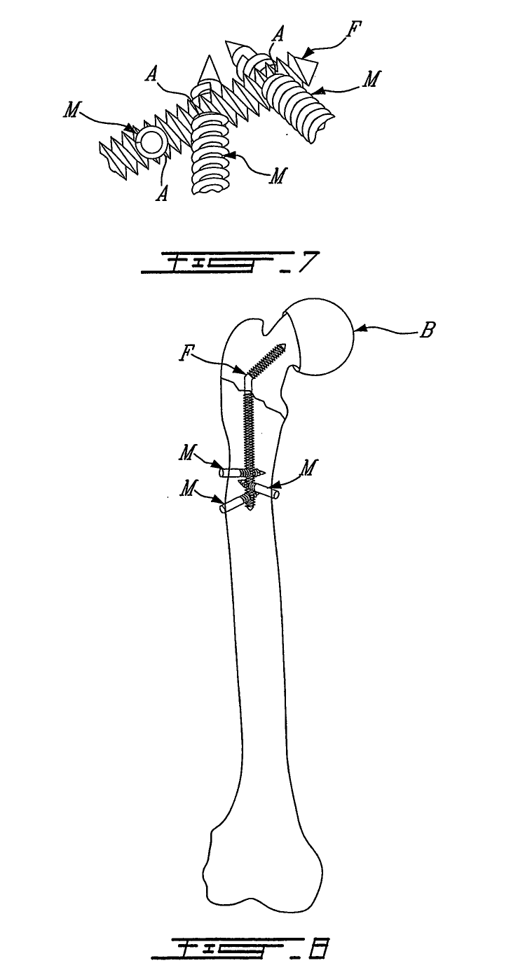

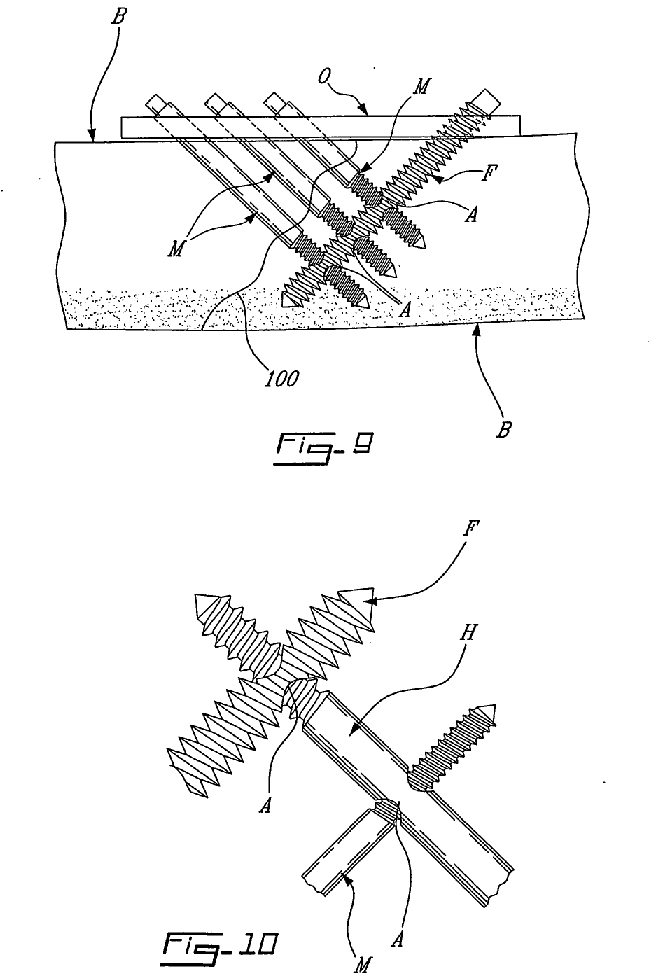

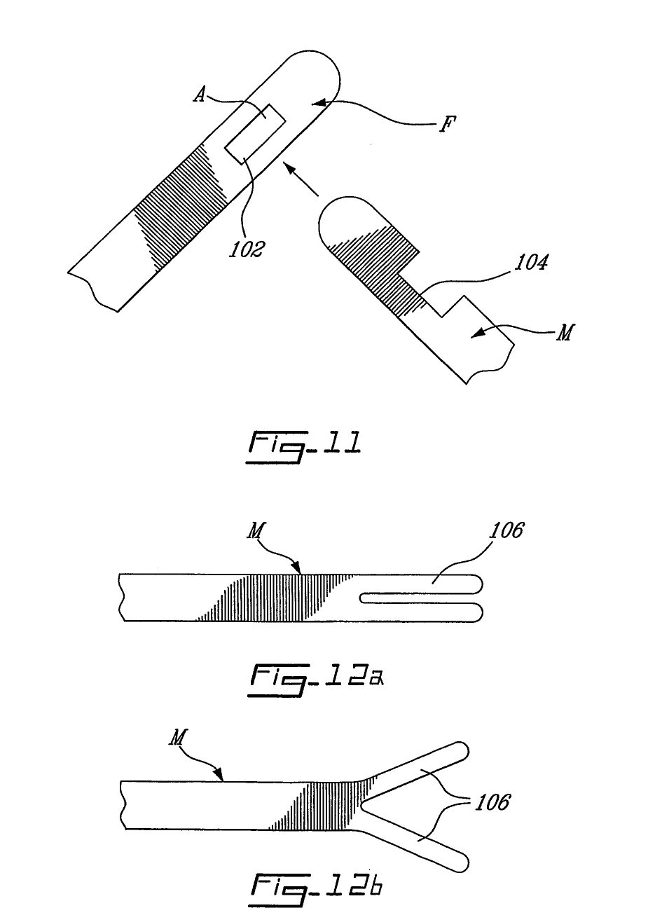

FIG. 1 illustrates an anchoring system S in accordance with the present invention which is herein schematically shown in an engaged position to a lumbar vertebra V of the human rachis for holding firmly thereagainst a spinal prosthesis or spinal instrumentation, such as a support plate P, from a posterior approach.

FIG. 2 illustrates two anchoring systems S that hold an object, such as a cervical plate P′ that has been positioned after classical anterior or antero-lateral approach of the cervical spine and that is herein used to link together two or more adjacent vertebrae, such as vertebrae V₁ and V₂. For instance, when a cervical disk is anteriorly removed (see 30 in FIG. 2) from between two adjacent vertebrae, it is known to fuse both these vertebrae together to provide stability to the rachis. This can be done by securing plates to the vertebrae. To replace the disks, a spinal prosthesis may be installed and such a prosthesis typically comprises a pair of upper and lower plates secured with screws respectively to the upper and lower vertebrae between which the disk has been removed with a prosthetic disk being provided between these vertebrae and which is held in place by the plates. U.S. Pat. No. 5,258,031 issued to Salib et al. on Nov. 2, 1993 and U.S. Pat. No. 6,001,130 issued to Bryan et al. on Dec. 14, 1999 disclose known examples of intervertebral disk prostheses which are secured to adjacent vertebrae using screws which extend therein. In these cases, one screw is used to mount each of the upper and lower support assemblies to its respective vertebra. On the other hand, U.S. Pat. No. 5,755,796 issued to Ibo et al. on May 26, 1998 teaches a cervical disk prosthesis wherein each of the upper and lower plates are secured to its respective vertebra by way of a pair of screws which are horizontally spaced apart from each other. The prosthesis is secured anteriorly of the rachis and the screws thereof are short enough not to reach the spinal cord.

In other cases however (posterior approach), the screws are longer and are generally directed on each side of the spinous canal in order to obviously prevent damage to the spinal cord.

Therefore, the present anchoring system S (for each of FIGS. 1 and 2) comprises first and second screws 10 and 12, respectively, which are adapted to be introduced in the vertebra at an angle and convergingly towards each other, as seen in FIG. 1. In the illustrated embodiment, the first screw 10 is larger than the second screw 20 and defines near its distal end an oblique threaded through opening 12. The two screws 10 and 20 have flat head 14 and 24 which define a depression which is shaped to be engageable by a screwdriver, or the like, for inducing torque thereto and causing the screws 10 and 20 to rotatably engage the vertebra V and gradually advance translationally thereinto, in a conventional manner. In FIG. 1, these depressions are slots 16 and 26 for use with a flat blade screwdriver, although the depressions could be cruciform, square, hexagonal, torx, etc., shaped.

The second screw 20 has a threaded stem of which at least a distal section is smaller than that of the first screw 10 as the second screw 20 is adapted to extend through the opening 12 of the first screw 10 such as to threadably engage the same. Indeed, the males threads of the second screw 20 are designed to engage the female threads of the opening 12 of the first screw 10 thereby securing together the distal ends of the two screws 10 and 20. With these distal ends so engaged and with the screws 10 and 20 extending in a convergent attitude, there is resistance, where the two screws 10 and 20 are engaged together, to the forces which tend to cause the screws to gradually loosen, whereby it is virtually impossible for the screws 10 and 20 to loosen (unless the vertebra V itself is destructed where it is engaged by the screws 10 and 20, or unless one of the screws 10 and 20 breaks).

In fact, the first screw 10 acts as a nut for the second screw 20, and this within the vertebra V itself in FIGS. 1 and 2 (as opposed to conventional nuts which normally engage the screw or bolt on the outside of the object through which the screw or bolt extends.

The first screw 10 is preferably provided at its head 14 with indicia (colour, index, etc.) to indicate the position of its distal end so that the position or orientation of its threaded opening 12 can be more easily determined thereby facilitating the introduction of the second screw 20 therethrough. An aiming system may be used as a guide during the screwing process. For instance, to ensure an accurate aiming of the two screws 10 and 20 and their relative engagement, a template may be used to guide both screws from an initial predetermined spacing, along a given plane and along predetermined angles. Alternatively, a neuro-navigation apparatus can also be used, that is a computer software capable of transposing digitised data taken from a pre-surgery medical imagery of the stereotactic space in which the surgeon will operate.

The obliqueness of the threaded opening 12 through the first screw 10 depends on the angle, that is on the spacing on the plate P/P′ between the two screws 10 and 20 (i.e. generally the spacing between their heads 14 and 24) in a horizontal plane, as well as the directions of the screws 10 and 20 in the sagittal plane.

The two screws 10 and 20 extend in holes defined in the plate P/P′, and would normally have their heads 14 and 24 in abutment with the proximal surface of the plate P/P′ (as in FIG. 2, but as opposed to the schematic illustration of FIG. 1 where the heads 14 and 24 are shown spaced from the plate P but simply for illustration purposes). The holes in the plate P/IP are typically angled so as to ensure the crossing of the screws 10 and 20 at a precise location in the vertebra V and so permit the threaded engagement of the second screw 20 in the opening 12 of the first screw 10 once the first screw 10 is completely fixed (i.e. screwed in the vertebra V) and once the position of its opening 12 is determined by way of the indicia on its head 14.

The two screws 10 and 20 and the plate P/P′ define a triangular frame (which is well shown in FIG. 1) which is rigid, closed and locked in place, having its components locked together in a solid medium, i.e. the vertebra V, whereby expulsion of the screws 10 and 20 from the vertebra V is opposed. Each of the three components 10, 20 and P/P′ of this frame is integral to the preceding component and to the next component. The triangulation screwing process is a concept based on the principle that a frame is much stronger than an open structure. By connecting two screws at their distal ends, it becomes possible to create such a frame. This triangular configuration is also convenient as it allows the anchoring system S, in addition to firmly securing the plate P/P′ to the vertebrae V, V₁ and V₂ and preventing a loosening of the plate P/P′ with respect to these vertebrae, to extend around the spinal process or canal C and thus around the spinal-cord when the plate P/P′ is, for instance, installed posteriorly (see FIG. 1).

In the case of the use of the anchoring system S to install the plate P′ onto the adjacent vertebrae V₁ and V₂ of the cervical rachis (FIG. 2), along an antero-lateral path, for instance following the removal of an herniated disc, osteosynthesis can be realised by fixing a plate P′ (e.g. a “Senegas”-type plate) with anchoring two systems S, as in FIG. 2. The plate P′ is centered about the intersomatic space 30, which is devoid of its natural disc, the latter having been replaced by a disc prosthesis. The first screws 10 are then positioned in the left holes 14 of the plate P′, along an antero-posterior axis or slightly obliquely from the left to the right, as the screwdriver will be hindered by the thickness of the oseo-tracheal axis (displaced to the left), before the first screws 10 are screwed through the left holes 14 and into the vertebrae V₁ and V₂. Once the first screws are completely set into the vertebrae V₁ and V₂, and properly positioned using their indicia, the two second screws 20 can be screwed through the right holes 14 of the plate P′ and into the vertebrae V₁ and V₂, which is easier than for the first screws 10 as the second screws 20 can be more easily inclined from right to left as the jugulo-carotid bundle is not as obstructive. A scopic control can ensure the proper engagement of the two screws 10 and 20 of each anchoring system S.

In the case of the plate P of FIG. 1 secured posteriorly at least to the vertebra V with the anchoring system S, the determination of the entry points in each of the pedicles of the vertebra V can be realised according to Roy-Camille. The plate P, or a linking rod, is then positioned horizontally and transversely such that its holes are opposite the pre-determined entry points. The screws 10 and 20 are then installed as above to form with the aforementioned triangular frame. This triangular frame, which is rigid and intra-vertebral, can then be solidified to upper and lower frames using plates or rods, in a conventional manner.

In order to facilitate the engagement of the second screw 20 into the first screw 10, the opening 12 in the first screw 10 may, instead of being threaded, have the form of a spherical socket that rotatably accommodates a ball. A hole extends, typically diametrically, through this ball and defines an interior thread, that is a female thread that can be screwably engaged by the male thread of the second screw 20. Therefore, the ball could rotate within the socket to allow for a correction in the direction of the second screw 20 relative to the first screw 10; in other words, if the second screw 20 is slightly off target in its orientation with respect to the hole defined in the ball of the first screw 10, the ball may be slightly rotated to align the longitudinal axis of its hole with the axis of the second screw 20.

It is also contemplated to provide a threadless opening in the first screw 10 instead of the threaded opening 12; in such a case, the opening would be self-tapping in that the male threads of the second screw 20 would tap a thread in the opening of the first screw 10 upon rotary engagement therein. Similarly, the above-mentioned ball could also be threadless and self-tapping. Furthermore, the first screw 10 could be replaced by a threadless pin or nail that would be translationally insertable in the bone and that would define an opening (threaded or self-tapping) at its distal end for receiving the second screw 20.

It is further contemplated to use elongated anchoring members other than the above-described and herein illustrated screws 10 and 20, as well as other means of securing the distal ends of such anchoring members together. For instance, the screws 10 and 20 could be replaced by threadless pins or nails that would be translationally inserted in the bone. In such a case, the distal end of a first one of the anchoring members could define an opening, such as an elongated slot, through which the distal end (which would, for instance, be flat) of a second one of anchoring members could be inserted. A locking mechanism between the two distal ends could take the form of a lateral pin extending from the distal end of the second anchoring member which, after having been passed beyond the elongated slot in the first anchoring member, would be rotated ¼ turn such as to extend behind the body of the first anchoring member thereby locking the distal ends together. Such a pin could be embodied in the distal end of the second anchoring member being L-shaped or T-shaped or defining a barb-shaped extension.

The common feature is two elongated members insertable in the bone and having distal ends capable of being interlocked for preventing unwanted withdrawal of any of the two elongated members from the bone.

Although the present anchoring system S has been shown herein in use to secure a plate P/P′ to one or more lumbar (FIG. 1) or cervical (FIG. 2) vertebrae, the system S can also be used to secure rods instead of plates, for instance to the dorso-lumbar rachis, and in fact can be used to affix various objects to various bones of the body, and not only to the rachis. The system S can thus be used not only as described above and herein illustrated, but also in orthopaedic, in neuro-surgical, otorhinolaryngological, maxillo-facial and stomatological applications.

Every component of the anchoring system S is made of a biocompatible material or of a material capable of being so coated.

In addition to the above general features and arrangements, various other designs and parameter modifications are contemplated to meet different needs.

In the following embodiments, the references “M” and “F” will be used to identify respectively male and female anchors (such as the first “female” and second “male” threaded fasteners of FIGS. 1 and 2) of different anchoring systems. These male and female anchors M and F can take different forms, such as the previously described threaded or threadless fasteners provided with different means to secure them together in the bone(s). The various openings in the anchors will be identified by reference “A”, whereas the objects that may be held to the bone via the anchors M and F will be identified by reference “O”.

For example, FIG. 3 illustrates the basic arrangement of the male and female anchors M and F of FIGS. 1 and 2, and wherein an angle α (above 0° and under 90°) is defined therebetween. In FIG. 4, the opening A in the female anchor F is positioned more proximally than in FIG. 3, that is the crossing distance d is less than in FIG. 3. Such parameters may depend on which type of bone and where in such a bone is the anchoring system being installed.

In FIG. 5, more than one male anchor M is used, namely three (3), and each male anchor M engages the same female anchor F. In FIGS. 3 to 5, the male and females anchors M and F extend coplanarly.

In FIGS. 6 and 6a, there is shown a bone B (e.g. a femur) that is broken at break 100, with the female anchor F having been positioned within the bone such as to extend through the break 100 for holding the bone sections together. The male anchors M are engaged in the female anchor F, and the whole arrangement of the male and female anchors M and F and of the object O forms a solid frame that not only brings the bone sections together but also maintains them in compression thereby promoting fusion thereof. The female anchor F transfixes the bone B while the male anchors M provide a return force (or a bias) that permits such compression.

It is noted that the object O through which the male anchors extend, outwardly of the bone B, can be a plate, a ring or washer, and the object O in some arrangements (e.g. depending on the shape of the heads of the male anchors M) can be omitted altogether, and this possibility applies to other configurations of the anchoring systems of the present invention.

FIGS. 7 and 8 are similar to FIGS. 5 and 6, except that the male anchors M in FIGS. 7 and 8 extend in different planes.

In FIG. 9 (using an arrangement similar to FIG. 5), the male anchors M extends through the break 100 in the bone B with the female anchor F being on an opposite side of the break 100 than the proximal ends of the male anchors M (and in this case of the object O, although the same is not always required). With this arrangement, there is a tightening under compression of the focus of the break 100. The male anchors M in some cases would not be coplanar.

The various anchoring mechanisms utilizing at least two male anchors M (e.g. the systems shown in FIGS. 5 to 9) can be useful, as mentioned above to stabilize a broken bone B, for instance a long bone (e.g. femur, humerus, cubitus, etc.) and to not only bring the various bone parts together but also to maintain them in a compressed state to facilitate the obtention of a callus and to thereby promote a fusion of the bone parts. The various male anchors M, extending coplanarly or not, assists in resisting axial torsion forces (the upper bone part could tend to rotate axially with respect to the lower bone part, in FIGS. 6 and 8, thereby necessitating a blocking via a third male anchor, and perhaps additional ones).

In the case of a multi-fragmentary bone fracture, the plurality of anchor members M can be used to bring together the various bone parts. As in FIGS. 6 and 8, the female anchor F is used internally of the bone B to transfix the bone parts by extending substantially parallel, or as parallelly as possible, through the focus of the break 100, and multiple male anchors M are connected to the female anchor F, either in a same plane (in the manner of a nail plate) or in different planes, if required. The male anchors M each have a flat head or are provided with a ring (or washer) to prevent them from digging into the bone B. The male members M can be perpendicular to the female member F. The screwed engagement of the male members will thus cause a return movement of the distal bone fragment engaged by the female anchor F that is engaged by the male anchors M, thereby ensuing an osteosynthesis under compressive forces.

FIG. 10 illustrates an anchoring system that includes an hybrid anchor H, i.e. an anchor that acts as a male anchor in engageably penetrating the female anchor F and as a female in having its aperture A engaged by the male anchor M.

The advantages of using such an hybrid anchor H are numerous: universal guiding system; single “screw-cotter pin”; alternate assembly that ensures a better cohesion of the structure.

FIGS. 11 to 13 show various arrangements that permit the male and female anchors M and F to be connected together. In FIG. 11, the female anchor F has its opening A in the form of a slot 102 adapted to received therethrough the male anchor M disposed in a proper orientation. The male anchor M defines a notch 104 that, once the male anchor M has been properly positioned through the female anchor F (i.e. with the notch 104 located within the slot 102) permits the male anchor M to be rotated ¼ of a turn thereby locking the male and female anchors M an F together.

In FIGS. 12a and 12b, the distal end of the male anchor M defines a fork 106 that can be displaced from a first “installation” position shown in FIG. 12a wherein the fork 106 of the male anchor M can be introduced through the opening A defined in the female anchor F, to a second “engagement” position shown in FIG. 12b wherein the fork 106 of the male anchor M is spread out so as to prevent removal of the male anchor M from the female anchor F. This arrangement basically operates like an umbrella. A material having a memory can be used to ensure the spreading of the fork 106 when it is released, i.e. after it has extended past the opening A (not shown) of the female anchor F.

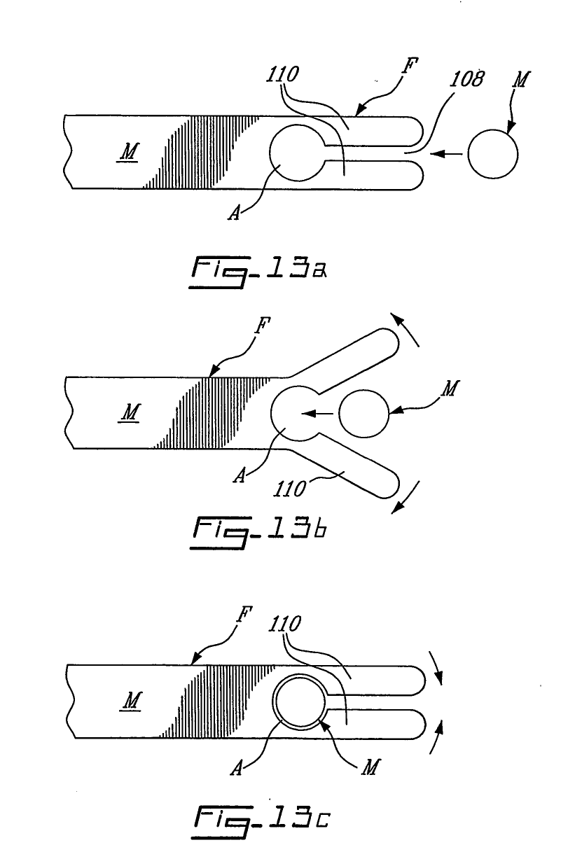

In FIGS. 13a to 13c, the distal end of the female anchor F is open via a slot 108 that communicates with the opening A, thereby forming a clip 110, which is adapted to resiliently be spread, as seen in FIG. 13b, when the male anchor M is forced therein, and to return to is drawn position (seen in FIG. 13c) once the male anchor M is within the opening A of the female anchor F, thereby maintaining the male and female anchors M and F connected together.

1-18. (canceled)

19. An anchoring system for emplacement in bone, comprising a female anchoring member and a plurality of male anchoring members, each anchoring member having a proximal end and a distal end, the plurality of male anchoring members each having a first adaptation to facilitate a mechanical connection to the female anchoring member and the female anchoring member having a plurality of second adaptations to facilitate mechanical connection to the plurality of male anchoring members, the proximal end of the female anchoring member being spaced apart from the proximal ends of the plurality of male anchoring members, whereby the plurality of male anchoring members form at least one acute angle with the female anchoring member when the female anchoring member and the plural male anchoring members are connected.

20. An anchoring system as defined in claim 19, wherein the plurality of male anchoring members extend coplanarly when connected to the female anchoring member.

21. An anchoring system as defined in claim 19, wherein the plurality of male anchoring members extend in at least two different planes when connected to the female anchoring member.

22. The anchoring system of claim 19, wherein the first adaptation is an external screw thread on the plurality of male anchoring members and the second adaptation is at least one mating tapped internal screw thread on the female anchoring member.

23. The anchoring system of claim 19, wherein the first adaptation is a hook provided on the plurality of male anchoring members and the second adaptation is at least one slot defined through the female anchoring member, the hook is adapted to be inserted through the at least one slot and to be rotated so as to engage the hook behind the female anchoring member.

24. An anchoring system as defined in claim 19, wherein the second adaptations is at least one opening defined in the female anchoring member, and the first adaptation is an expendable element provided on the plurality of male anchoring members and adapted to be inserted through the at least one opening and to spread once behind the female anchoring member.

25. An anchoring system as defined in claim 24, wherein the expendable element provided on the plurality of male anchoring members spreads automatically once behind the female anchoring member.

26. An anchoring system as defined in claim 24, wherein the expendable element provided on the plurality of male anchoring members is to be spread mechanically once behind the female anchoring member.

27. An anchoring system as defined in claim 19, wherein the second adaptation is at least one clip provided on the female anchoring member, and adapted to displace from a male anchoring member receiving position for receiving said male anchoring member to a male anchoring member retaining position for connecting said male and female anchoring members together.

28. An anchoring system as defined in claim 19, wherein the plurality of male anchoring members include a first male anchoring member having an opening adapted to be engaged by a further male anchoring member, the first male anchoring member thereby acting as a hybrid anchoring member that both engages the female anchoring member and is engaged by the further male anchoring member.

29. An anchoring system as defined in claim 19, wherein the female anchoring member extends within a bone and through a break defined therein, the plurality of male anchoring members engaging the female anchoring member for maintaining the bone together.

30. An anchoring system for mounting an object to a bone, comprising a female anchoring member and a plurality of male anchoring members, each anchoring member having a proximal end and a distal end, the proximal ends being adapted to hold the object to the bone, the plurality of male anchoring members each having a first adaptation to facilitate a mechanical connection to the female anchoring member and the female anchoring member having a plurality of second adaptations to facilitate mechanical connection to the plurality of male anchoring members, the proximal end of the female anchoring member being spaced apart from the proximal ends of the plurality of male anchoring members, whereby the plurality of male anchoring members form at least one acute angle with the female anchoring member when the female anchoring member and the plural male anchoring members are connected.

31. An anchoring system as defined in claim 30, wherein the plurality of male anchoring members extend coplanarly when connected to the female anchoring member.

32. An anchoring system as defined in claim 30, wherein the plurality of male anchoring members extend in at least two different planes when connected to the female anchoring member.

33. The anchoring system of claim 30, wherein the first adaptation is an external screw thread on the plurality of male anchoring members and the second adaptation is at least one mating tapped internal screw thread on the female anchoring member.

34. The anchoring system of claim 30, wherein the first adaptation is a hook provided on the plurality of male anchoring members and the second adaptation is at least one slot defined through the female anchoring member, the hook is adapted to be inserted through the at least one slot and to be rotated so as to engage the hook behind the female anchoring member.

35. An anchoring system as defined in claim 30, wherein the second adaptations is at least one opening defined in the female anchoring member, and the first adaptation is an expendable element provided on the plurality of male anchoring members and adapted to be inserted through the at least one opening and to spread once behind the female anchoring member.

36. An anchoring system as defined in claim 30, wherein the second adaptation is at least one clip provided on the female anchoring member, and adapted to displace from a male anchoring member receiving position for receiving said male anchoring member to a male anchoring member retaining position for connecting said male and female anchoring members together.

37. An anchoring system as defined in claim 30, wherein the plurality of male anchoring members include a first male anchoring member having an opening adapted to be engaged by a further male anchoring member, the first male anchoring member thereby acting as a hybrid anchoring member that both engages the female anchoring member and is engaged by the further male anchoring member.
